(19) United States
(12) Patent Application Publication
(10) Pub. No.: US 2008/0280989 A1
(43) Pub. Date: Nov. 13, 2008

(54) Hydroxybenzamide Derivatives, the Method For Preparing Thereof and the Cosmetic Composition Containing the Same

(76) Inventors: Sung Joong Kim, Ansan-si (KR); Heung Soo Baek, Seoul (KR); Ho Sik Rho, Yongin-si (KR); Duck Hee Kim, Seoul (KR); Ih Seop Chang, Yongin-si (KR); Ok Sub Lee, Anyang-si (KR); Hong Ju Shin, Seongnam-si (KR); Woo Ram Park, Yongin-si (KR)

Correspondence Address: NIXON & VANDERHYE, PC, 901 NORTH GLEBE ROAD, 11TH FLOOR, ARLINGTON, VA, 22203, US

(21) Appl. No.: 11/990,223

(22) PCT Filed: Mar. 20, 2006
(86) PCT No.: PCT/KR2006/001006

§ 371 Date: Mar. 24, 2008

(30) Foreign Application Priority Data
Aug. 19, 2005 (KR) 10-2005-0076184

(51) Int. Cl. A61K 31/165; C07C 233/07; A61P 17/00

(52) U.S. Cl. 514/617; 564/179

(57) ABSTRACT

Disclosed is a hydroxybenzamide derivative represented by the following Formula. A method for preparing the same and a cosmetic composition comprising the same are also disclosed. More particularly, the hydroxybenzamide derivative is obtained by reacting a hydroxybenzoic acid having a protecting group introduced thereto with a hydroxyphenyl amine to form a benzamide derivative and by hydrolyzing the benzamide derivative in an aqueous base solution to form a hydroxybenzamide derivative. The cosmetic composition comprising the hydroxybenzamide derivative as an active ingredient has excellent anti-oxidative, anti-aging and skin wrinkle-alleviating effects. wherein R1 represents a C1˜C10 alkyl group, and n is an integer ranging from 1 to 3.

### TECHNICAL FIELD

The present invention relates to a hydroxybenzamide derivative represented by the following Formula 1, a method for preparing the same, and a cosmetic composition comprising the same. More particularly, the present invention relates to a hydroxybenzamide derivative obtained by reacting a hydroxybenzoic acid having a protecting group introduced thereto with a hydroxyphenyl amine to form a benzamide derivative and by hydrolyzing the benzamide derivative in an aqueous base solution to form a hydroxybenzamide derivative, as well as to a cosmetic composition comprising the hydroxybenzamide derivative as an active ingredient and having excellent anti-oxidative, anti-aging and skin wrinkle-alleviating effects.

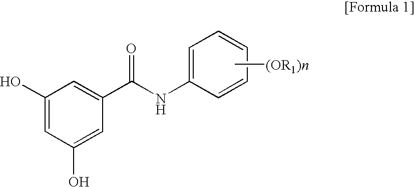

wherein R₁ represents a C1˜C10 alkyl group, and n is an integer ranging from 1 to 3.

### BACKGROUND ART

Resveratrol is a kind of phytoalexin, which is a material produced by some plants for the purpose of self-protection. It is known that resveratrol has the effect of preventing cardiac diseases and cancers derived from inhibition of coagulation of blood platelet, prevention of lipid protein oxidation and reduction of fatty acids, while showing the effects of wrinkle alleviation, whitening, anti-oxidation, anti-aging, anti-inflammation and anti-irritation in the skin cells (Chem. Pharm. Bull. 2002, 50(4), 450; Free Radicial Biology & Medicine 2002, 33(8), 1089; Thrombosis Research 2002, 106, 205; Chem. Eur. J. 2002, 8(18), 4191; Toxicology and Applied Pharmacology 2003, 186, 28). However, despite such various favorable effects, there has been a limitation in use of resveratrol as a cosmetic agent due to its low stability. Therefore, there has been a need for a cosmetic agent which can improve stability of resveratrol while maintaining the effects thereof.

### DISCLOSURE

### Technical Problem

Therefore, the present invention has been made in view of the above-mentioned problems. The inventors of the present invention have conducted intensive studies to solve the problems occurring in resveratrol, including structural deformation in a formulation containing resveratrol, and thus have developed a hydroxybenzamide derivative having excellent stability while maintaining the known effects of resveratrol, including skin wrinkle-alleviating and anti-oxidative effects. This results in completion of the present invention.

Therefore, an object of the present invention is to provide a novel hydroxybenzamide derivative as a derivative of resveratrol, and a method for preparing the same.

Another object of the present invention is to provide a cosmetic composition comprising the above hydroxybenzamide derivative and having excellent anti-oxidative, anti-aging and skin wrinkle-alleviating effects.

### Technical Solution

According to an aspect of the present invention, there is provided a hydroxybenzamide derivative represented by the following Formula 1:

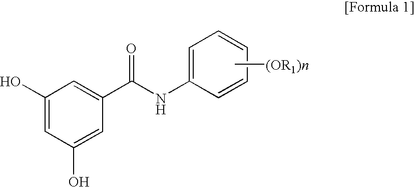

wherein R₁ represents a C1˜C10 alkyl group, and n is an integer ranging from 1 to 3.

According to another aspect of the present invention, there is provided a method for preparing the hydroxybenzamide derivative represented by the above Formula 1, the method comprising the steps of: reacting a hydroxybenzoic acid having a protecting group introduced thereto with a hydroxyphenyl amine in an organic solvent to form a benzamide derivative; and deprotecting the benzamide derivative in an aqueous base solution to form the hydroxybenzamide derivative represented by Formula 1. Also, there is provided a cosmetic composition comprising the hydroxybenzamide derivative represented by Formula 1 as an active ingredient.

Hereinafter, the present invention will be explained in more detail.

The method for preparing a hydroxybenzamide derivative represented by Formula 1 comprises the steps of:

(i) introducing a protecting group into a hydroxyl group of 3,5-dihydroxybenzoic acid;
(ii) reacting the benzoic acid having a protecting group introduced thereto, obtained from step (i), with a hydroxyphenylamine in the presence of methanesulfonyl chloride to form a hydroxyphenylbenzamide; and
(iii) deprotecting the hydroxyphenylbenzamide obtained from step (ii) in an aqueous base solution to form a derivative represented by Formula 1.

Also, the method for preparing a hydroxybenzamide derivative may be represented by the following Reaction Scheme 1:

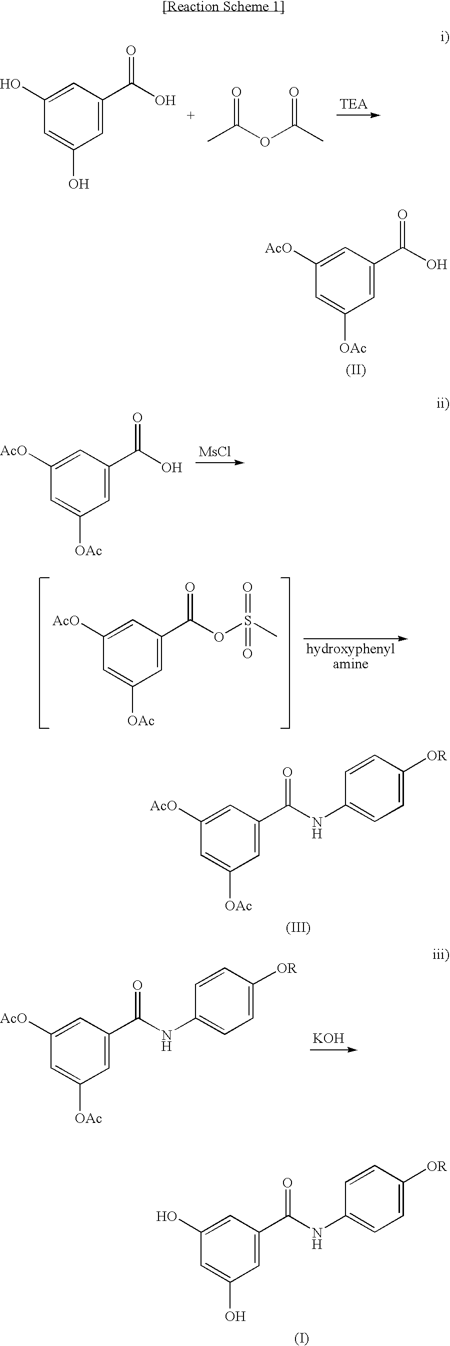

Hereinafter, the processing steps as shown in Reaction Scheme 1 will be explained in more detail.

(i) Step of introducing a protecting group into 3,5-dihydroxybenzoic acid to form diacetyloxybenzoic acid represented by Formula II.

Particular examples of the protecting group used in this step include methyl ether, ethyl ether, benzyl ether, formate acetate, benzoate ester, acetate ester, or the like. Acetate ester is the most preferred. In this step, pyridine, triethylamine (TEA), etc. may be used as an organic base, and dichloromethane, chloroform, tetrahydrofuran, etc. may be used as an organic solvent.

Additionally, the reaction is performed at a temperature of 10˜80° C., preferably of 40° C.

In one embodiment of the reaction, 3,5-dihydroxybenzoic acid (15.4 g, 0.09 mol), triethylamine (45 ml, 0.32 mol) and 4-dimethylaminopyridine (0.1 g, 0.0008 mol) are added to 150 ml of tetrahydrofuran. Next, acetic anhydride (30 ml, 0.31 mol) is added dropwise thereto under reflux to obtain 3,5-diacetyloxybenzoic acid (Formula II) into which an acetyl protecting group is introduced.

(ii) Step of reacting the compound represented by Formula II with a hydroxyphenyl amine in the presence of methanesulfonyl chloride to form diacetyloxyu-N-hydroxyphenylbenzamide (Formula III).

Although the compound represented by Formula III may be prepared by way of the acid halogenation method, active ester method, acid anhydride method, or the like, it is the most preferred that the compound of Formula III is prepared by reacting a hydroxyphenyl amine with an active ester using methanesulfonyl chloride. Also, in this step, pyridine, triethylamine, etc. may be used as an organic base, triethylamine being preferred. Additionally, dichloromethane, chloroform, tetrahydrofuran, etc., may be used as an organic solvent.

Particular examples of the hydroxyphenyl amine that may be used in this step include 4-aminophenol, 2-aminophenol, 3-aminophenol, p-anisidine, 3,4-dimethoxyaniline, 3,5-dimethoxyaniline, 3,4,5-trimethoxyaniline, 5-amino-2-methoxyphenol, or the like, but are not limited thereto.

In one embodiment of the reaction, 3,5-diacetyloxybenzoic acid (23.8 g, 0.1 mol) and triethylamine (15 ml, 0.107 mol) are added to 200 ml of tetrahydrofuran, and methanesulfonyl chloride (8 ml, 0.103 mol) is added dropwise thereto. The reaction mixture is stirred for 30 minutes, filtered under reduced pressure to remove triethylamine salt, and added dropwise to 4-aminophenyl (12 g, 0.109 mol) in tetrahydrofuran (100 ml). The reaction mixture is stirred for 3 hours, and recrystallized in 10% aqueous ethanol solution to obtain white diacetyloxy-N-hydroxyphenylbenzamide (Formula III).

(iii) Step of deprotecting the compound of Formula III obtained from step (ii) in an aqueous base solution to form a hydroxyphenylbenzamide (Formula I).

In this step, an alkali metal hydroxide such as sodium hydroxide or potassium hydroxide may be used as the base. Additionally, as the reaction solvent, water, methanol, ethanol, a mixed solvent of methanol with tetrahydrofuran or water with tetrahydrofuran, or the like may be used. Among these solvents, water is the most preferred.

In one embodiment of the reaction, 3,5-diacetyl-N-hydroxyphenylbenzamide (Formula III) (8 g, 0.024 mol) is added to 0.5M aqueous potassium hydroxide solution (300 ml), and the reaction mixture is refluxed for 30 minutes. Next, 1M aqueous HCl solution is added to the reaction mixture to acidify the mixture to a pH value of 4˜3. Then, the resultant white precipitate is filtered under reduced pressure, and washed with water many times to obtain a hydroxyphenyl benzamide (Formula I).

Preferred examples of the hydroxybenzamide derivative according to the present invention include:
3,5-dihydroxy-N-(4-hydroxyphenyl)benzamide;
3,5-dihydroxy-N-(2-hydroxyphenyl)benzamide;
3,5-dihydroxy-N-(3-hydroxyphenyl)benzamide;
3,5-dihydroxy-N-(4-methoxyphenyl)benzamide;
3,5-dihydroxy-N-(3,4-dimethoxyphenyl)benzamide;
3,5-dihydroxy-N-(3,5-dimethoxyphenyl)benzamide;
3,5-dihydroxy-N-(3,4,5-trimethoxyphenyl)benzamide; and
3,5-dihydroxy-N-(3-hydroxy-4-methoxyphenyl)benzamide.

The hydroxybenzamide derivative represented by Formula 1, obtained by the method according to the present invention, shows high stability in an aqueous or organic solvent, while exhibiting excellent skin wrinkle-alleviating and anti-oxidative effects. Thus, the hydroxybenzamide derivative according to the present invention may be applied to cosmetic compositions for alleviating skin wrinkles and cosmetic compositions having an anti-aging effect, besides conventional cosmetic compositions.

### BEST MODE FOR CARRYING OUT THE INVENTION

Reference will now be made in detail to the preferred embodiments of the present invention. It is to be understood that the following examples are illustrative only and the scope of the present invention is not limited thereto.

### EXAMPLE 1

### Preparation of 3,5-dihydroxy-N-(4-hydroxyphenyl)benzamide

### Preparation of 3,5-diacetyloxybezoic acid: Reaction Scheme 1-i

To 200 ml of tetrahydrofuran, 15.4 g (0.09 mol) of 3,5-dihydroxybenzoic acid and 38 ml (0.27 mol) of triethylamine were added, and the reaction mixture was stirred for 10 minutes. To the reaction mixture, 23 ml (0.24 mol) of acetic anhydride was added dropwise, and then the resultant mixture was refluxed for 3 hours. The reaction mixture was cooled to room temperature and allowed to evaporate under reduced pressure. Then, dichloromethane and water were added thereto, and the organic layer was washed with water and 1N aqueous HCl solution many times, and allowed to evaporate under reduced pressure. Hexane was added to the remaining oil to form precipitate. The precipitate was filtered under reduced pressure to obtain 15 g (90%) of the target product.

### Preparation of 3,5-diacetyloxy-N-(4-hydroxy phenyl)benzamide: Reaction Scheme 1-ii

To 200 ml of tetrahydrofuran, 11.9 g (0.05 mol) of 3,5-diacetyloxybenzoic acid and 8 ml (0.05 mol) of triethylamine were added, and the reaction mixture was cooled to 0° C. To the reaction mixture, 4 ml (0.05 mol) of methanesulfonyl chloride was added dropwise, and the reaction mixture was stirred for 20 minutes. Next, 6 g (0.05 mol) of 4-aminophenol was added thereto. The reaction mixture was stirred for 4 hours while maintaining the reaction temperature at 0° C., and was allowed to evaporate under reduced pressure. A small amount of ethanol was added to the remaining product to dissolve it, 0.5N aqueous HCl solution was added thereto, and the reaction mixture was stirred rigorously to form white precipitate. The precipitate was filtered under reduced pressure to obtain 13 g (80%) of the target product.

### Preparation of 3,5-dihydroxy-N-(4-hydroxyphenyl)benzamide: Reaction Scheme 1-iii

First, 8 g (0.024 mol) of 3,5-diacetyloxy-N-(4-hydroxyphenyl)benzamide was added to 300 ml of 0.5M aqueous potassium hydroxide solution, and the reaction mixture was refluxed for 40 minutes. The solution was cooled to room temperature, and 1N aqueous HCl solution was added thereto to acidify the solution to a pH value of 4˜3. The resultant white precipitate was filtered under reduced pressure and washed with water many times to obtain 6 g (90%) of the pure target product.

¹H-NMR (300 MHz, DMSO-d₆): δ 9.8 (s, 1H), 9.5 (s, 2H), 9.2 (s, 1H), 7.5 (d, 2H), 6.7 (m, 4H), 6.3 (s, 1H).

### EXAMPLE 2

### Preparation of 3,5-dihydroxy-N-(2-hydroxyphenyl)benzamide

The target product was obtained in an amount of 4 g (70%) by using the same method as described in Example 1, except that 2-aminophenol was used instead of 4-aminophenol in step (ii) of Example 1.

¹H-NMR (300 MHz, DMSO-d₆): 10.2 (s, 1H), 9.8 (bs, 3H), 7.4 (d, 1H), 7.3 (t, 1H), 6.9 (m, 3H), 6.7 (d, 1H), 6.5 (s, 1H).

### EXAMPLE 3

### Preparation of 3,5-dihydroxy-N-(3-hydroxyphenyl)benzamide

The target product was obtained in an amount of 4.5 g (75%) by using the same method as described in Example 1, except that 3-aminophenol was used instead of 4-aminophenol in step (ii) of Example 1.

¹H-NMR (300 MHz, DMSO-d₆): 10.2 (s, 1H), 9.8 (bs, 3H), 7.4 (d, 1H), 7.0 (m, 2H), 6.9 (m, 3H), 6.7 (s, 1H).

### EXAMPLE 4

### Preparation of 3,5-dihydroxy-N-(4-methoxyphenyl)benzamide

The target product was obtained in an amount of 4.5 g (75%) by using the same method as described in Example 1, except that p-anisidine was used instead of 4-aminophenol in step (ii) of Example 1.

¹H-NMR (300 MHz, DMSO-d₆): 8.0 (s, 1H), 7.5 (d, 2H), 6.9 (s, 2H), 6.7 (d, 2H), 6.4 (s, 1H), 5.0 (bs, 2H), 3.7 (s, 3H).

### EXAMPLE 5

### Preparation of 3,5-dihydroxy-N-(3,4-dimethoxyphenyl)benzamide

The target product was obtained in an amount of 4.8 g (70%) by using the same method as described in Example 1, except that 3,4-dimethoxyaniline was used instead of 4-aminophenol in step (ii) of Example 1.

¹H-NMR (300 MHz, DMSO-d₆): 10.2 (s, 1H), 9.8 (bs, 2H), 7.2 (s, 1H), 7.0 (d, 1H), 6.9 (m, 3H), 6.5 (s, 1H), 3.8 (s, 3H), 3.7 (s, 3H).

### EXAMPLE 6

### Preparation of 3,5-dihydroxy-N-(3,5-dimethoxyphenyl)benzamide

The target product was obtained in an amount of 4.5 g (75%) by using the same method as described in Example 1, except that 3,5-dimethoxyaniline was used instead of 4-aminophenol in step (ii) of Example 1.

¹H-NMR (300 MHz, DMSO-d₆): 8.0 (s, 1H), 6.9 (s, 2H), 6.7 (s, 2H), 6.4 (s, 1H), 6.0 (s, 1H), 5.0 (bs, 2H), 3.7 (s, 6H).

### EXAMPLE 7

### Preparation of 3,5-dihydroxy-N-(3,4,5-trimethoxyphenyl)benzamide

The target product was obtained in an amount of 4.5 g (60%) by using the same method as described in Example 1, except that 3,4,5-trimethoxyaniline was used instead of 4-aminophenol in step (ii) of Example 1.

¹H-NMR (300 MHz, DMSO-d₆): 10.0 (s, 1H), 9.7 (bs, 2H), 7.0 (s, 2H), 6.7 (s, 2H), 6.5 (s, 1H), 3.7 (m, 9H).

### EXAMPLE 8

### Preparation of 3,5-dihydroxy-N-(3-hydroxy-4-methoxyphenyl)benzamide

The target product was obtained in an amount of 4.6 g (70%) by using the same method as described in Example 1, except that 5-amino-2-methoxyphenol was used instead of 4-aminophenol in step (ii) of Example 1.

¹H-NMR (300 MHz, DMSO-d₆): 10.2 (s, 1H), 9.8 (bs, 3H), 7.0 (m, 4H), 6.8 (d, 1H), 6.4 (s, 1H), 3.8 (s, 3H).

### TEST EXAMPLE 1

### Determination of Anti-Oxidative Effect Using HaCat Model

The hydroxybenzamide compounds prepared in Examples 1-8 were determined for their anti-oxidative effects.

Human keratinocyte HaCaT cell lines were pipetted into 60 mm dishes in a cell count of 1.0×10⁶ cells per dish, and were cultured by using a DMEM (FBS 10%) medium containing penicillin/streptomycin added thereto under the conditions of 37° C./5% CO₂ for 1 day. Then, the cultured product was treated with each of the hydroxybenzamide compounds according to Examples 1˜8 in a concentration of 10˜4 mol. Also, the same cultured product was treated with the same concentration of tocopherol and resveratrol for 24 hours. In addition to the above, the cultured product was treated with t-BHT (t-butyl hydroperoxide) and cultured under the conditions of 37° C./5% CO₂ for 4 hours to obtain cells. The cells were subjected to lysis by repeating freezing/thawing cycles. The following Test procedure was based on the method described in the assay kit used in this example.

In this example, Calbiochem Lipid peroxidation assay kit (Cat. No. 437634) was used as a test reagent, and lipid peroxidation was determined by using the mechanism of formation of stable compounds at 586 nm from the reaction between the above reagent and ester peroxides linked to long-chain unsaturated fatty acids, such as malondialdehyde (MDA) and 4-hydroxyalkenal (4-hydroxy-2(E)-nonenal, 4-HNE).

TABLE 1

| Sample | Lipid peroxidation (%) |
|---|---|
| Non-treated group | 100 |
| t-BHT | 320 |
| Tocopherol | 250 |
| Resveratrol | 178 |
| Ex. 1 | 170 |
| Ex. 2 | 180 |
| Ex. 3 | 182 |
| Ex. 4 | 186 |
| Ex. 5 | 176 |
| Ex. 6 | 172 |
| Ex. 7 | 179 |
| Ex. 8 | 181 |

As can be seen from the above results listed in Table I showing the anti-oxidative effect of each sample, each of the hydroxybenzamide compounds according to Examples 1˜8 shows a higher anti-oxidative effect as compared to the positive control, tocopherol, while showing a similar anti-oxidative effect as compared to resveratrol.

### TEST EXAMPLE 2

### Stimulation of Collagen Biosynthesis

The hydroxybenzamide compounds according to Examples 18 were determined for their effects of stimulating collagen biosynthesis, and the results were compared to the effects obtained from resveratrol and tocopherol.

Fibroblasts were seeded into a 24-well microtiter plate in a cell count of 10⁵ cells per well and cultured to a growth level of 90%. The cultured product was further cultured in a serum-free DMEM medium for 24 hours. Next, the cultured product was treated with the hydroxybenzamide compounds according to Examples 1˜8, resveratrol and tocopherol, dissolved in a serum-free medium at a concentration of 10 μM, and then was cultured in a CO₂ incubator for 24 hours. After decanting the supernatant, procollagen was determined by using a procollagen type(I) ELISA kit. The results are shown in the following Table 2, wherein the biosynthesis activity is expressed based on the biosynthesis activity of non-treated group, taken as 100.

TABLE 2

| | Collagen biosynthesis |
|---|---|
| Sample | activity (%) |
| Non-treated group | 100 |
| Tocopherol | 113 |
| Resveratrol | 111 |
| Ex. 1 | 143 |
| Ex. 2 | 121 |
| Ex. 3 | 122 |
| Ex. 4 | 118 |
| Ex. 5 | 115 |
| Ex. 6 | 116 |
| Ex. 7 | 119 |
| Ex. 8 | 120 |

As can be seen from the results listed in Table 2 showing the effects of stimulating collagen biosynthesis, each of the hydroxybenzamide compounds according to Examples 1˜8 has an effect of stimulating collagen biosynthesis. It can be also seen that each of the compounds according to Examples 18 is superior to the positive controls, i.e. tocopherol and resveratrol, in terms of the effect of stimulating collagen biosynthesis.

### TEST EXAMPLE 3

### Determination of Effect of Inhibiting Collagenase Expression

The hydroxybenzamide compounds according to Examples 1˜8 were determined for their effects of inhibiting collagenase expression, and the results were compared to the effects obtained from resveratrol and tocopherol.

Human fibroblasts were introduced into a 96-well microtiter plate containing a DMEM (Dulbecco's Modified Eagle's Media) with 2.5% fetal bovine serum (FBS) in a cell count of 5,000 cells per well and cultured to a growth level of 90%. Then, the cultured product was further cultured in a serum-free DMEM medium for 24 hours. Next, the cultured product was treated with the hydroxybenzamide compounds according to Examples 18, resveratrol and tocopherol, dissolved in a serum-free DMEM medium at a concentration of 10˜4M, and then the cell culture was collected. The cell culture was evaluated for the production of collagenase by using a commercially available collagenase measuring system (Amersham Pharmacia, USA). First, the cell culture was introduced into a 96-well plate coated uniformly with primary collagenase antibodies, and then an antigen-antibody reaction was carried out in an incubator for 3 hours. After 3 hours, secondary collagenase antibodies, to which chromophores were bound, were introduced into the 96-well plate, and the reaction was further carried out for 15 minutes. After 15 minutes, a color developer was added thereto to develop a color at room temperature for 15 minutes, and 1M sulfuric acid was further added thereto to quench the reaction (color development). This resulted in development of a yellow color from the reaction mixture, wherein the yellowness varied with reaction degrees. The yellow-colored 96-well plate was measured for absorptivity at 405 nm by using an absorption spectrometer, and a degree of collagenase synthesis was calculated according to the following Mathematical Formula 1. At this time, absorptivity of the cell culture in a non-treated group was used as a control.

Collagenase expression (%)=Absorptivity of the group treated with the corresponding sample/Absorptivity of the control×100 [Mathematical Formula 1]

The following Table 3 shows the results of inhibition of collagenase expression in the cells. As can be seen from Table 3, the hydroxybenzamide compounds according to the present invention can inhibit collagenase expression in vitro. The collagenase expression inhibiting activity was expressed based on the collagenase synthesis activity of the non-treated group, taken as 100.

TABLE 3

| | Collagenase |
|---|---|
| Sample | expression (%) |
| Non-treated group | 100 |
| Tocopherol | 95 |
| Resveratrol | 81 |
| Ex. 1 | 67 |
| Ex. 2 | 69 |
| Ex. 3 | 71 |
| Ex. 4 | 74 |
| Ex. 5 | 72 |
| Ex. 6 | 71 |
| Ex. 7 | 70 |
| Ex. 8 | 69 |

As can be seen from the results listed in Table 3 showing the effects of inhibiting collagenase expression, each of the hydroxybenzamide compounds according to Examples 1˜8 has an effect of inhibiting collagenase expression.

### TEST EXAMPLE 4

### Determination of Thermal Stability According to Isothermal Discoloration Test

The hydroxybenzamide compounds according to Examples 18 were determined for their thermal stability as compared to resveratrol.

The test was performed by dissolving each of the hydroxybenzamide compounds according to Examples 1˜8 into a test solvent (dimethylformaldehyde:ethanol:water=5:3:2) in a concentration of 1000 pm, and by allowing the test samples to be left in an isothermal chamber at 40° C. for a test period of 30, 60 and 90 days. Then, the test samples were observed by the naked eyes to determine discoloration degrees.

Discoloration of the samples was graded into Grade 1 Grade 4 as follows:

1: no discoloration

2: discoloration into a light yellow color

3: discoloration into a dark yellow color

4: discoloration into a dark brown color

TABLE 4

| | Discoloration | Discoloration | Discoloration |
|---|---|---|---|
| Sample | after 30 days | after 60 days | After 90 days |
| Resveratrol | 2 | 3 | 4 |
| Ex. 1 | 1 | 1 | 1 |
| Ex. 2 | 1 | 1 | 1 |
| Ex. 3 | 1 | 1 | 1 |
| Ex. 4 | 1 | 1 | 1 |
| Ex. 5 | 1 | 1 | 1 |
| Ex. 6 | 1 | 1 | 1 |
| Ex. 7 | 1 | 1 | 1 |
| Ex. 8 | 1 | 1 | 1 |

As can be seen from the above results listed in Table 4, each of the hydroxybenzamide compounds according to Examples 1˜8 has excellent thermal stability as compared to resveratrol.

Based on the results obtained from the above Test Examples 1˜4, cosmetic preparations comprising a hydroxybenzamide compound represented by Formula 1, having excellent stability in a formulation containing the same, and showing excellent anti-aging and wrinkle-alleviating effects were prepared as Formulation Examples 1˜6. However, the following Formulation Examples are merely illustrative, and the scope of the present invention is not limited thereto.

### FORMULATION EXAMPLE 1

### Skin Toner

| Components | Wt % |
|---|---|
| Ex. 1 | 0.2 |
| Cholesterol | 0.7 |
| Glycerin | 3.0 |
| 1,3-butylene glycol | 1.0 |
| Cellulose gum | 0.1 |
| Ethanol | 10.0 |
| POE-16 octyl dodecyl ether | 0.2 |
| Polysorbate-60 | 0.2 |
| Preservative | trace amount |
| Pigment | trace amount |
| Perfume | trace amount |
| Purified water | balance |

### FORMULATION EXAMPLE 2

### Nourishing Toner

| Components | Wt % |
|---|---|
| Ex. 1 | 1.0 |
| Stearic acid | 0.7 |
| Cholesterol | 1.0 |
| Cetostearyl alcohol | 0.7 |
| Polysorbate-60 | 1.5 |
| Sorbitan sesquioleate | 0.5 |
| Liquid paraffin | 5.0 |
| Squalane | 5.0 |
| glycerine | 5.0 |
| Carboxyvinyl polymer | 0.1 |
| Triethanol amine | 0.12 |
| Preservative | trace amount |
| Pigment | trace amount |
| Perfume | trace amount |
| Purified water | balance |

### FORMULATION EXAMPLE 3

### Nourishing Cream

| Components | Wt % |
|---|---|
| Ex. 1 | 3.0 |
| Cholesterol | 5.0 |
| Cetostearyl alcohol | 3.0 |
| Stearic acid | 2.0 |
| Polysorbate-60 | 1.5 |
| Sorbitan sesquioleate | 0.5 |
| Liquid paraffin | 10.0 |
| Squalane | 10.0 |
| Glycerin | 6.0 |
| Triethanol amine | 0.5 |
| Preservative | trace amount |
| Pigment | trace amount |
| Perfume | trace amount |
| Purified water | balance |

### FORMULATION EXAMPLE 4

### Essence

| Components | Wt % |
|---|---|
| Ex. 1 | 1.0 |
| Myristic acid | 5.0 |
| Cholesterol | 7.0 |
| Cetostearyl alcohol | 1.0 |
| Glycerin | 15.0 |
| 1,3-butylene glycol | 4.0 |
| Cellulose gum | 0.1 |
| Hyaluronic acid extract | 10.0 |
| Carboxyvinyl polymer | 0.12 |
| Triethanol amine | 0.17 |
| Ethanol | 3.0 |
| Polysorbate-60 | 0.2 |
| POE-25 octyl dodecylether | 0.2 |
| Preservative | trace amount |
| Pigment | trace amount |
| Perfume | trace amount |
| Purified water | balance |

### FORMULATION EXAMPLE 5

### Cleansing Foam

| Components | Wt % |
|---|---|
| Ex. 1 | 2.0 |
| Cholesterol | 5.0 |
| Bees wax | 1.0 |
| Stearic acid | 5.0 |
| Polysorbate-60 | 0.5 |
| Myristic acid | 26.0 |
| Potassium hydroxide | 5.0 |
| Glycerin | 6.0 |
| EDTA-4 sodium | 0.2 |
| Pigment | trace amount |
| Perfume | trace amount |
| Purified water | balance |

### FORMULATION EXAMPLE 6

### Pack

| Components | Wt % |
|---|---|
| Ex. 1 | 3.0 |
| Cholesterol | 0.7 |
| Polyvinyl alcohol | 14.0 |
| Cellulose gum | 0.1 |
| Glycerin | 1.0 |
| PEG 4000 | 1.0 |
| POE-16 octyl dodecyl ether | 0.4 |
| Alcohol | 6.0 |
| Preservative | trace amount |
| Pigment | trace amount |
| Perfume | trace amount |
| Purified water | balance |

### INDUSTRIAL APPLICABILITY

As can be seen from the foregoing, the compound according to the present invention, the hydroxybenzamide derivative represented by the above Formula 1, shows high stability in an aqueous or organic solvent, as well as has excellent skin wrinkle-alleviating and anti-oxidative effects. Therefore, the hydroxybenzamide derivative according to the present invention can be applied to skin wrinkle-alleviating and anti-aging cosmetic compositions.

While this invention has been described in connection with what is presently considered to be the most practical and preferred embodiment, it is to be understood that the invention is not limited to the disclosed embodiment and the drawings. On the contrary, it is intended to cover various modifications and variations within the spirit and scope of the appended claims.

1. A hydroxybenzamide derivative represented by the following Formula 1: 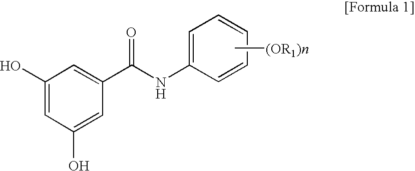
wherein R₁ represents a C1˜C10 alkyl group, and n is an integer ranging from 1 to 3.

2. A method for preparing the hydroxybenzamide derivative as defined in claim 1, the method comprising the steps of:
(i) introducing a protecting group into a hydroxyl group of 3,5-dihydroxybenzoic acid to form diacetyloxybenzoic acid compound;
(ii) reacting the benzoic acid having a protecting group introduced thereto, obtained from step (i), with a hydroxyphenylamine in the presence of methanesulfonyl chloride to form a hydroxyphenylbenzamide; and
(iii) deprotecting the hydroxyphenylbenzamide obtained from step (ii) in an aqueous base solution to form a hydroxybenzamide derivative.

3. The method according to claim 2, wherein the protecting group introduced in step (i) is selected from the group consisting of methyl ether, ethyl ether, benzyl ether, formate acetate, benzoate ester and acetate ester.

4. The method according to claim 2, wherein the hydroxyphenyl amine used in step (ii) is selected from the group consisting of 4-aminophenol, 2-aminophenol, 3-aminophenol, p-anisidine, 3,4-dimethoxyaniline, 3,5-dimethoxyaniline, 3,4,5-trimethoxyaniline and 5-amino-2-methoxyphenol.

5. A cosmetic composition comprising the hydroxybenzamide derivative as defined in claim 1 as an active ingredient.

6. A skin wrinkle-alleviating or anti-aging cosmetic composition comprising the hydroxybenzamide derivative as defined in claim 1 as an active ingredient.
